(19) United States
(12) Patent Application Publication
(10) Pub. No.: US 2022/0162841 A1
(43) Pub. Date: May 26, 2022

(54) INTELLIGENT NETWORKED TOILET SYSTEM WITH CUSTOMIZABLE FEATURE SET

(71) Applicant: Shine Bathroom Technologies, Inc., Santa Barbara, CA (US)

(72) Inventors: Chris Herbert, Santa Barbara, CA (US); John Stump, Santa Barbara, CA (US)

(21) Appl. No.: 17/536,015

(22) Filed: Nov. 27, 2021

Related U.S. Application Data
(63) Continuation of application No. 16/665,689, filed on Oct. 28, 2019.
(60) Provisional application No. 62/752,903, filed on Oct. 30, 2018.
(60) Provisional application No. 62/781,410, filed on Dec. 18, 2018.
(60) Provisional application No. 62/808,218, filed on Feb. 20, 2019.

(51) Int. Cl. E03D 9/00; H04L 67/125

(52) U.S. Cl. CPC E03D 9/002; E03D 2009/028; H04L 67/125

(57) ABSTRACT

Systems and methods that provide enhanced capability for toilets and bathrooms in general by adding feature sets such as user weighing, leak detection, enhanced toilet cleaning/deodorizing and other features, connected over networks to user devices and servers, creating a complete bathroom ecology where users have direct and remote control and access to system functions and data, and server based bathroom support applications provide a wide range of capabilities including user control and readout of toilet systems functions, bathroom condition, supply levels, and health data.

### RELATED APPLICATIONS

This application is Continuation of U.S. application Ser. No. 16/665,689, filed Oct. 28, 2019, which claims priority to U.S. Provisional Application Ser. No. 62/752,903 filed Oct. 30, 2018, Ser. No. 62/781,410 filed Dec. 18, 2018, and Ser. No. 62/808,218 filed Feb. 20, 2019, all of which are incorporated by reference in their entirety

### FIELD

The present disclosure generally relates to enhanced capability toilets and toilet accessories, and in particular to network connected toilet systems capable of remote control from user devices and in communication with server based support capability.

### BACKGROUND

Capabilities are becoming possible for traditional home devices such as toilets to add intelligence and connectivity, enabling rich feature sets and remote control from anywhere with network accessibility, creating in effect, a toilet system capable of performing a variety of useful functions ranging from health related operations to various ways of making the bathroom experience more pleasant. Moreover with certain types of sensors, actuators, and supply reservoirs, a networked toilet may report useful information about both the user and the toilet systems well as keep track of supply levels and even automatically order replenishments.

### SUMMARY

The systems and methods of this disclosure each have several innovative aspects, no single one of which is solely responsible for its desirable attributes. Without limiting the scope as expressed by the claims that follow, its more prominent features will now be discussed briefly.

Systems and methods may be provided that provide enhanced capability for toilets and bathrooms in general by adding feature sets such as user weighing, comfort features, leak detection, enhanced toilet cleaning/deodorizing and other features, connected over networks to user devices and servers, creating a complete bathroom ecology where users have direct and remote control and access to system functions and data, and server based bathroom support applications provide a wide range of capabilities including user control and readout of toilet systems functions, bathroom condition, supply levels, and health data.

In a first aspect, a remotely actuated electrolyzer may be provided, including; at least one reservoir comprising a control valve and a fill sensor, at least one access point for a replaceable active ingredient pod; at least one anode and cathode for electrolysis; a power supply; and, a processing and network interface element (i.e. controller) configured to: provide control signals to the pump, electrode and control valve, receive fill sensor data and at least one of; receive user ID information from one or more network sources, receive power supply status from the power supply, receive control information from a user, receive control information from the network, receive control information from a sensor, and actuate at least one of the pump, valve, and electrode in response to control information.

In a second aspect, a toilet cleaning system may be provided including the electrolyzer as above, and further including at least one pump, a feedline from the pump to a toilet, wherein the system elements are packaged together in a housing wherein the electrolyzer includes a base, a removable reservoir, and a pods/electrode mounting configured for access to the reservoir when all elements are assembled together.

In one embodiment of the second aspect, the system may report usage and when electrolysis was last done to a server. In another embodiment of the second aspect, the server may automatically reorder more cleaning pods based on usage data received. In one embodiment of the second aspect, the system may accept user commands from a mobile app, webpage, or API.

In another embodiment of the second aspect, the reservoir may be configured to at least one of; sit on floor adjacent toilet, hang from toilet body by means of a hanging bracket, mount to wall adjacent toilet, or sit on top of toilet, or placed on counter top or other fixed horizontal surface. In one embodiment of the second aspect, the active ingredient pod may include a foil sealed vessel, which when installed in the reservoir the foil may be pierced by a pin allowing the active ingredients to drain into the reservoir. (note pod can be sealed with foil or other polymer material) In another embodiment of the second aspect, the processor may accept and provide information across the network with user devices including Personal Electronic Devices running applications specific to the toilet cleaning system.

In one embodiment of the second aspect, the feedline may terminate in a sprayer attachable to the toilet bowl. In another embodiment of the second aspect, the sprayer may at least one of clips, sticks via adhesives, held by suction, or mechanically connected to the toilet. In one embodiment of the second aspect, the sprayer may include three nozzles at differing angles to deliver a fine spay mist wherein the mist may cover the entire inside of the toilet bowl, wherein to deliver the fine mist, the fluid moves through tubing, through the sprayer, into the nozzle cavity, around a pin, through texture at the tip of the pin, and finally exiting through the nozzles.

In another embodiment of the second aspect, the sprayer may be user adjustable to adjust the spray via the user interface, to change firmware to increase or decrease power to the pump wherein lower power results in more of a stream of fluid for more of a point contact of the fluid to the bowl and higher power creates more pressure resulting in a fine mist. In one embodiment of the second aspect, the power supply may include at least one of; a battery, or a plug in power source.

In another embodiment of the second aspect, the system may further include a flow (flush) sensor mountable onto a water line including a toilet feedline and in wired or wireless communication with the toilet system processor, wherein the processor is configured to selectively trigger the pump, valves, and electrode upon flush detection. In one embodiment of the second aspect, the flow sensor may include an accelerometer, packaged in a housing configured to clamp around a water line, including the toilet feedline and wired to the system electronics for power and signals.

In another embodiment of the second aspect, the system may include at least one Internet server configured as a toilet system service provider for one of tracking or automatically ordering supplies based on toilet system use. In one embodiment of the second aspect, the base may include at least one of a power inlet and an external data interface including USB variants. In another embodiment of the second aspect, the system may include a nightlight, controllable by one of user commands, network command, or internal timer.

In one embodiment of the second aspect, the installation of a pod may be electrically communicated to the processor. In another embodiment of the second aspect, the processor may at least one of send an alert or flash an LED after a predetermined time after pod installation to replace pod In one embodiment of the second aspect, the system may include at least one Internet server configured as a toilet system service provider wherein at least one of the server or processor sends an alert after a predetermined time after pod installation to replace pod. In another embodiment of the second aspect, the system may detect leak conditions by means of the flow sensor and may communicate leak conditions by way of sending an alert.

In one embodiment of the second aspect, the system may include at least one Internet server configured as a toilet system service provider, wherein the server is configured to communicate to the user at least one of, water usage, estimated water cost, toilet use, estimate the associated consumables around toilet usage such as toilet paper and hand soap, compare water usage to usage in local area, leak profile, suggested repairs, or send user a repair kit. In another embodiment of the second aspect, the system may include a database containing leak patterns, toilet models, and location information where leak data collected from the system can be compared with leak data on the database to determine the most likely cause of the leak. In one embodiment of the second aspect, the system may include the use of a machine learning algorithm to pattern match the historical water information collected from the system to precisely determine the cause of the leak.

### BRIEF DESCRIPTION OF THE DRAWINGS

The above-mentioned aspects, as well as other features, aspects, and advantages of the present technology will now be described in connection with various implementations, with reference to the accompanying drawings. The illustrated implementations are merely examples and are not intended to be limiting. Throughout the drawings, similar symbols typically identify similar components, unless context dictates otherwise.

FIG. 1 illustrates a general arrangement of an example networked toilet system.

FIG. 2 shows elements of an exemplary weighing system.

FIGS. 3A, 3B and 3C show an exemplary weight measurement function.

FIG. 4 shows elements of an exemplary heating system.

FIG. 5 shows elements of an exemplary leak/flush detection and position sensing.

FIG. 6 shows elements of an exemplary network interfaced electrolyzing system.

FIG. 7 shows elements of an exemplary network interfaced electrolyzing system as part of a toilet leaning system.

FIGS. 8A, 8B, 8C, and 8D illustrate various exemplary mounting and packaging options for a toilet cleaning system.

FIGS. 9A and 9B illustrate various exemplary toilet cleaning system elements.

FIG. 10 illustrates more detailed exemplary toilet cleaning system elements.

FIGS. 11A and 11B illustrate various exemplary electrolysis additive pod and reservoir elements.

FIGS. 12A and 12B illustrate various exemplary flow/flush sensor elements.

FIGS. 13A and 13B illustrate various exemplary sprayer elements.

FIGS. 14A and 14B illustrate various exemplary sprayer details.

FIGS. 15A, 15B, and 15C illustrate various exemplary sprayer spray patterns.

FIG. 16 illustrates various exemplary sprayer and nozzle embodiments.

FIGS. 17A, 17B, and 17C illustrate various exemplary nozzle embodiments.

FIGS. 18, 19, and 20 illustrate various exemplary server based operational modes.

### DETAILED DESCRIPTION

The following description is directed to certain implementations for the purpose of describing the innovative aspects of this disclosure. However, a person having ordinary skill in the art will readily recognize that the teachings herein can be applied in a multitude of different ways.

Generally described, embodiments of the present disclosure relate to network interfaced devices and systems aimed at improving the functionality and convenience of bathroom use. These devices may in many embodiments center around the toilet, as bathroom use, consumption of supplies and health related data all may be derived from monitoring various aspects of toilet use. Network interfaces allow both the control and data presentation to occur via the use of ubiquitous Personal Electronic Devices (PED's) such as smartphones, smartwatches, tablets and personal computers. In the most complete implementation, the toilet related elements, the user devices and application services running on network servers provide a complete highly automated bathroom ecology providing health and use tracking, maintenance and repair services, and remote control of many common bathroom functions, all centered around smart, networked toilet centric elements.

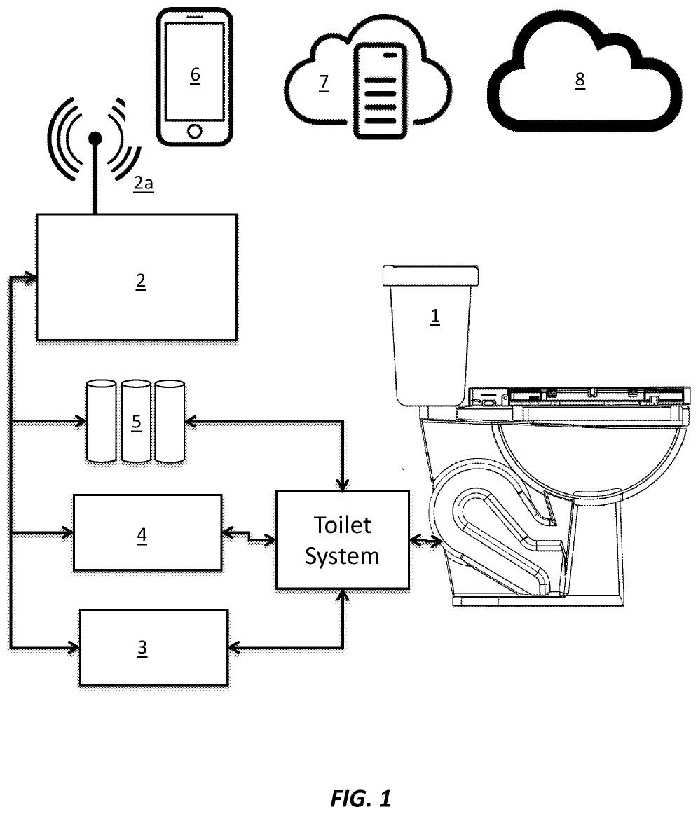

The general features of such toilet elements, 1, are shown in FIG. 1. Inherent is a processor/network interface 2. The processor interfaces to a variety of toilet elements that taken together or in part add a variety of features to the base toilet. These elements among others may include sensors 3, actuators 4 and reservoirs for various additive substances 5. The Processor/network interface 2, otherwise referred to as the controller 2, may execute software routines, logic sequences and/or other programmed functions that through the use of the controllable elements create enhanced capabilities.

Although wired or wireless user interfaces from toilet specific remote control units are functional, it is contemplated that the controller 2 for many embodiments communicate wirelessly 2a through any combination of short range local networks (e.g. Bluetooth, Zigbee and the like) and/or medium range local networks (e.g. WiFi etc.), and will in most embodiments included direct and/or through a bridging device connection to the open internet. And it is also contemplated that user interface, automatic control functions, data collection and logging, as well as resource tracking and even supply ordering will take place on devices running toilet system applications including user Personal Electronic Devices 6 (PED's), e.g. smartphones, tablets, PC's etc.), Dedicated toilet system servers 7 providing services such as data logging and processing, supply ordering. etc., and other general cloud based 8 resources. The toilet system power supply 9 could be a plug in system utilizing home AC, or it could be a battery.

We will now discuss a particular intelligent, networked toilet system application which is based on a capability to determine a user's weight while using the toilet system.

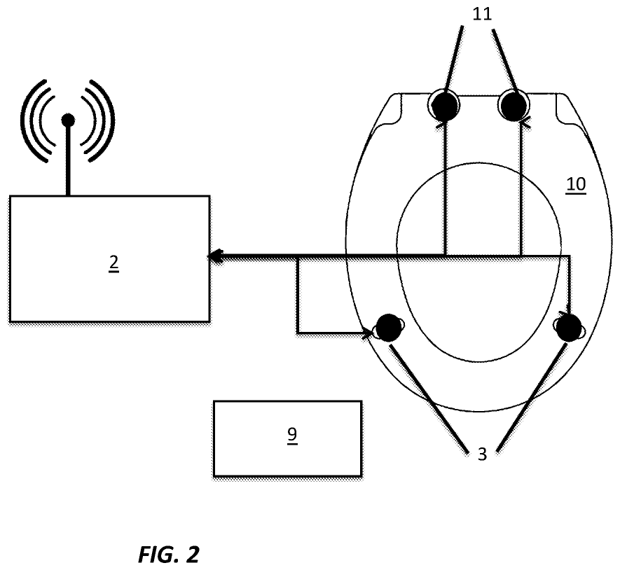

Referring to FIG. 2 a toilet seat 10 is shown, wherein weight sensors 11 are load sensors, such as suitably sized load cells. Load cells of a size that will fit within a typical toilet seat envelope can be found in the 50 to a few hundred pound range, so a beneficial number to cover a wide range of user weights as well as support a symmetrical arrangement is four cells. It has been found beneficial to mount the load cells with two taking the place of front seat support bumps and two at the hinge posts, as shown in the Figure. This arrangement provides for a floating suspension beneficial for a weighing operation. Other configurations such as having 6-12 contact mounts and/or more contact points, may improve accuracy regarding leaning, or other variations in posture that may make it difficult to obtain accurate weigh analysis, as will be described below.

As described above, the weight sensors 11 interface to the controller which can provide weight information and user interface functions across the network interface to either user devices in proximity, such as user PED's 6 over a network such as Bluetooth as well as provide data to and receive data from servers 7 on the Internet (cloud 8), as well as other devices over the internet.

As the normal user position while using a toilet does not support all of a user's weight on the seat, it may be desirable to calibrate for this affect.

A calibration algorithm may translate the weight detected by the toilet seat when someone is sitting on the seat to their actual real weight. Unlike regular chairs where users sit on the chair in a variety of positions, a toilet seat has only a few variations of ways to sit on it. Combined with the fact that since using the toilet is a task performed in relatively the same way, every day, Muscle Memory theories take effect. An algorithm may benefit from repetitive behavior to determine the user's weight distribution of weight on seat vs weight on feet when sitting on the toilet. The algorithm achieves this by conducting a calibration over several days to determine what is the average % weight on the seat vs the floor.

The algorithm begins with a user taking measurements at known weight distributions. The user will be instructed to take these weight measurements via instructions delivered via an application (app) with text, images, video, and/or other means using the user device accelerometer. In this example implementation, the user would take 3 weight measurements, but it would be possible to increase or decrease the number of weight measurements for less accuracy or more accuracy respectively.

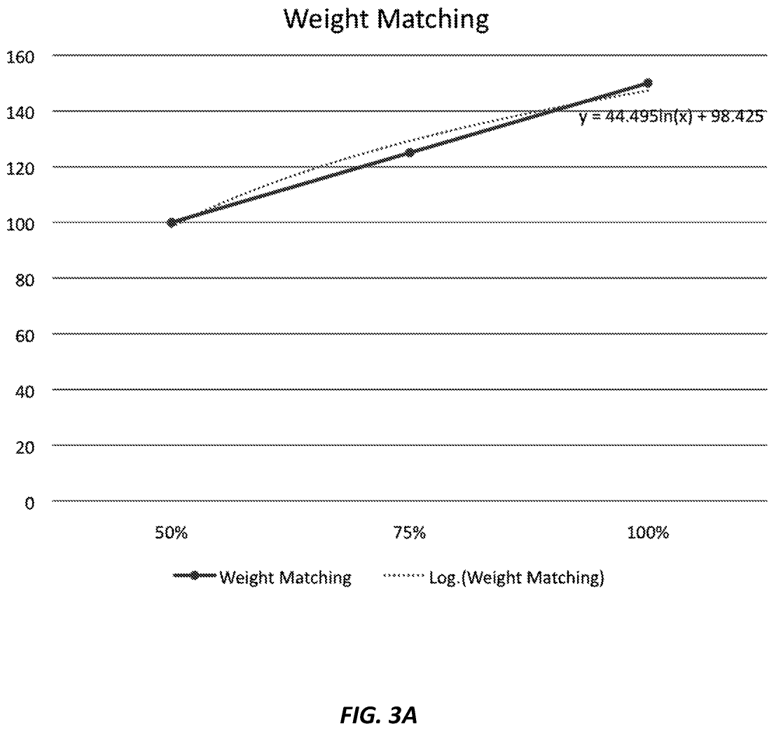

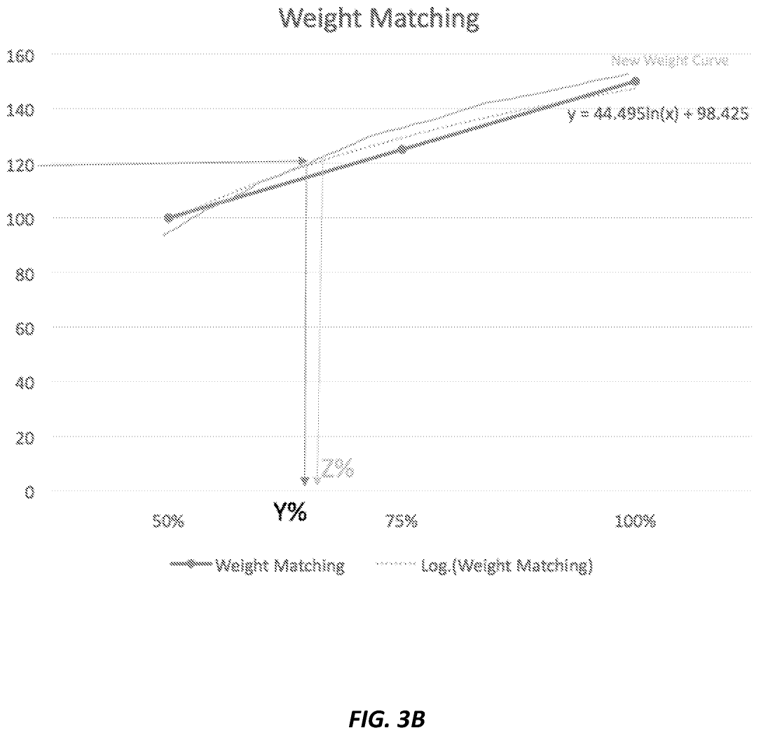

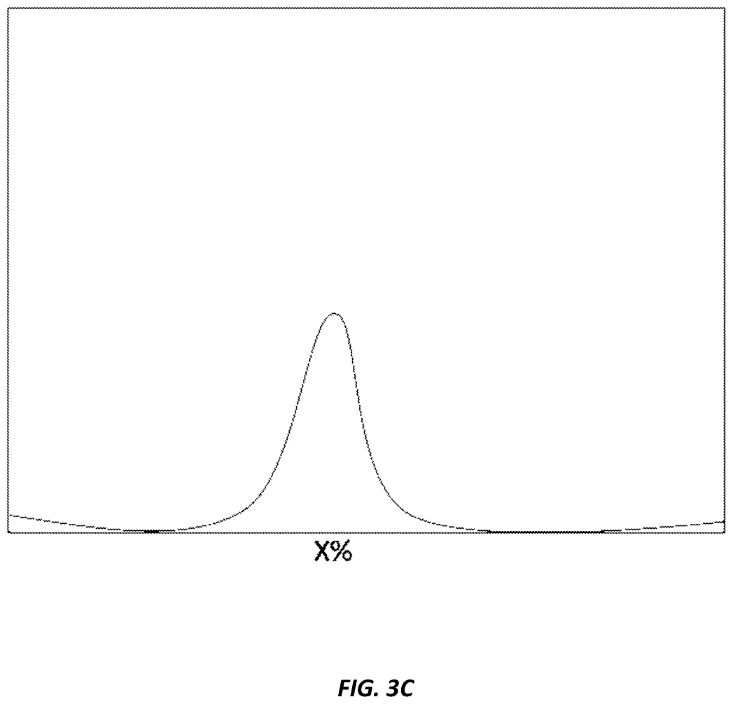

As shown in FIG. 3A, the 3 weight measurements, can be matched to a predictive line curve derived from previous general measurements to correlate the weight to certain percentage weight distributions.

The next time of use the user sits down and the toilet seat captures the user weight. The weight is then matched to a Y % via the curve originally derived. This % is recorded in a database stored on system or in the cloud.

Referring to FIG. 3B, to increase accuracy the user is then instructed lift their feat to capture the 100% real weight. The weight curve is then redrawn and with the new 100% weight and shifting the other 2 matching weight curves by a factor of the (new 100% weight)/(old 100% weight). The user measured weight is then correlated to a new percent Z % and this is stored in a database. This discovery process is repeated for every use until a statistically significant amount of % points have been collected in order to determine the weight % distribution.

Referring to FIG. 3C, graphing the %'s recorded from the database, one can calculate the mode, median, average, and standard deviation on the weight % distribution for that user. Using these functions and the graph, one skilled in the art can derive a predictive algorithm to calculate the users % distribution when sitting on the toilet. This % can then be used to calculate the user's actual weight when sitting on the toilet with a certain known % accuracy.

Other seat related functions could be integrated conveniently in an instrumented seat, as shown in FIGS. 4 and 5. Heating elements 13 may be desirable and may benefit from the use of a gasket 12 between heating layers and the rest of the seat structure. Position sensors 16 to determine if the seat is open or shut could be present and interfaced to the controller 2. A flow sensor 15 to determine when flushing takes place may also be desirable, as will be described in more detail below.

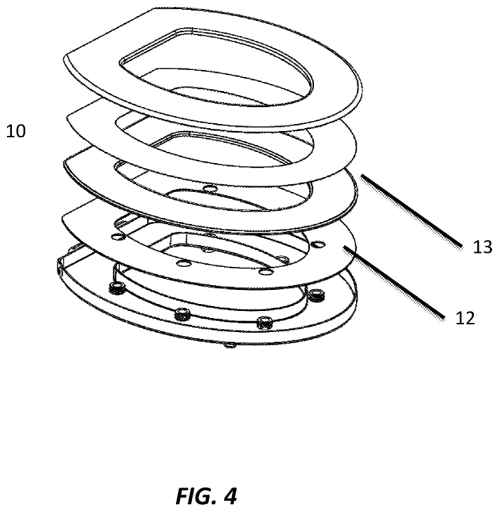

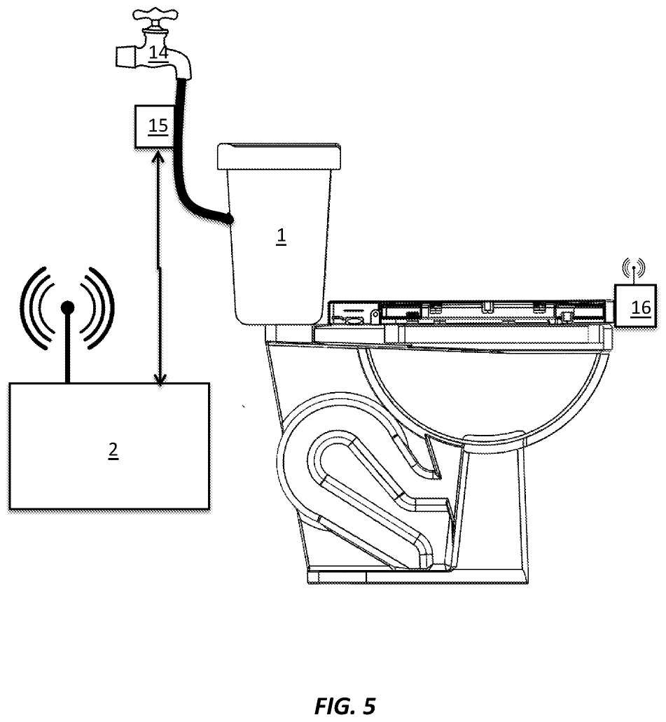

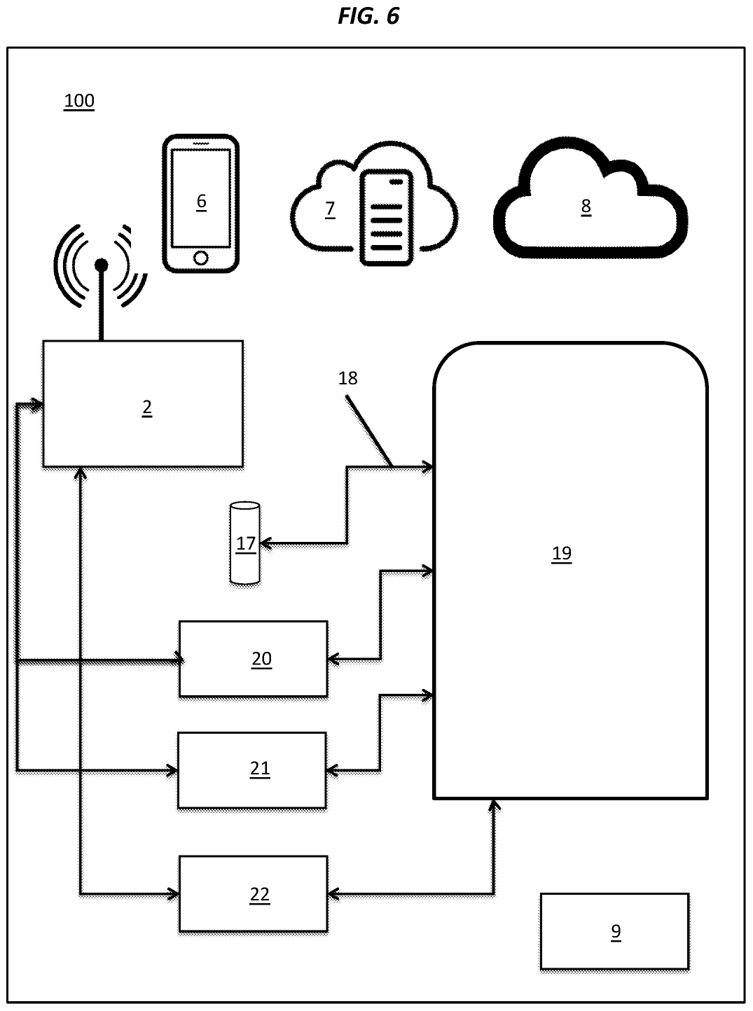

FIG. 6 shows the elements of a networked electrolyzing system 100, an important part of a smart toilet cleaning system, as well as applicable to other uses. Controller 2 interfaces to PED's 6, and cloud 8 based servers 7. Also possible are local dedicated user interfaces such as screens, and buttons, but such local interfaces may not be needed for most implementations. Reservoir 19 is a fluid container. In a likely implementation, reservoir 19 is Tillable with locally available water, such as tap water of possibly easily available purified water. In this case to increase the effectiveness of electrolyzed water as a cleaner, a removable pod 17 of electrolyte may be attachable to a pod port 18, serving as a replaceable, renewable source of electrolytes for the reservoir. Electrodes 21 (Anode and cathode) are arranged to contact reservoir 19 fluid, and be actuated under controller 2 direction. In the spirit of the electrolyzer being network connected and remotely controlled, optional elements 20, for example such as fill sensors and control valves, and pump 22 also interfaced to the controller may be present. For some applications a hand pump may be of use, but an electrically actuated pump is likely in the automated toilet cleaning implementation. Power supply 9 is also a likely feature and will be discussed more below in the context of the toilet cleaning device. Thus the electrolyzer 100 shown, under controller and often network control, electrolyzes water on command, and the water is enriched with a replaceable pod of additives.

FIG. 7 shows additional elements, flow sensor 15, attachable to a waterline/source such as a toilet supply line 14 and sprayer 23 connected by a feedline to the electrolyzer 100, in particular to the pump, and disposed to be attachable to toilet 1 in a manner that will spray electrolyzed water into the toilet bowl for cleaning. Adding these elements to electrolyzer 100 under control of controller 2 produce a smart toilet cleaning system.

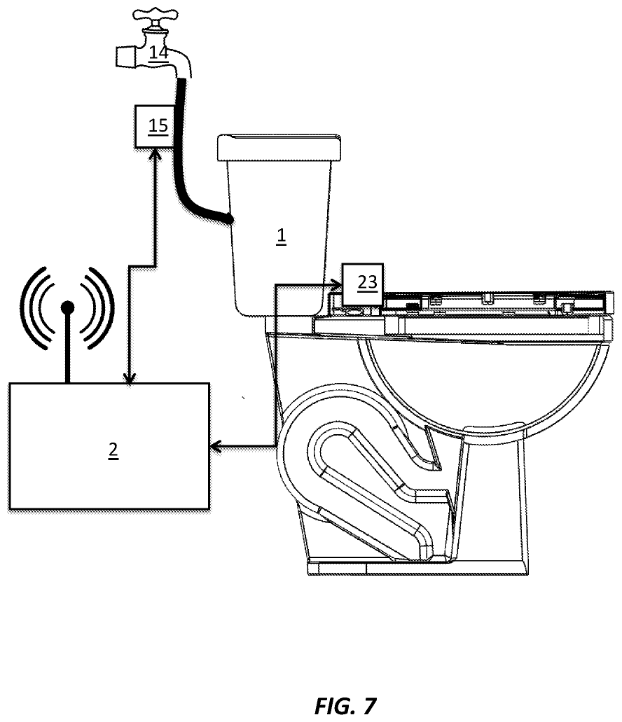

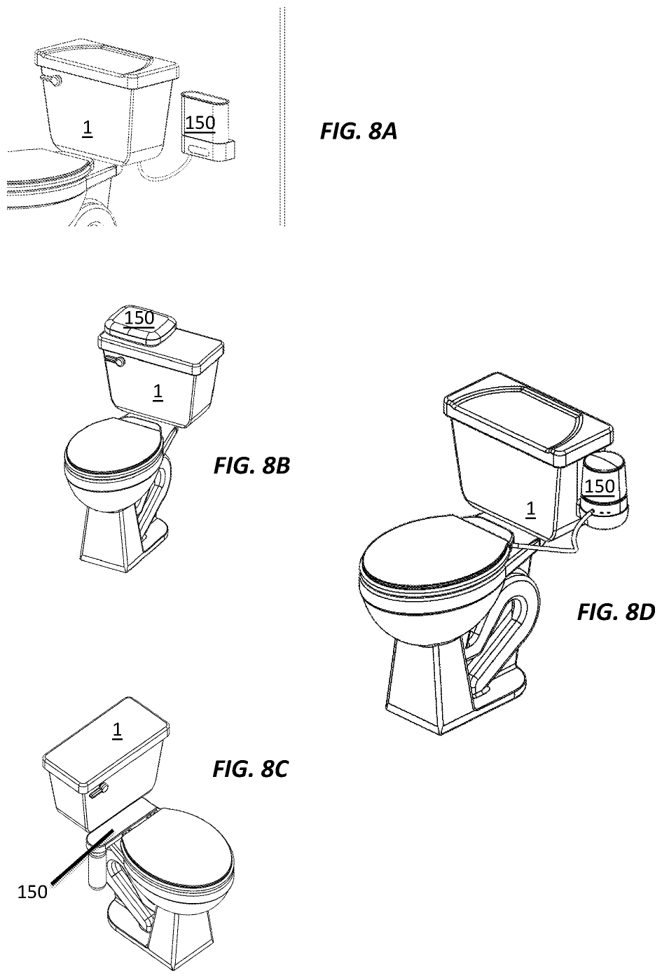

In many embodiments, it may be desirable to implement cleaning system 150 as an integrated unit, with a flow sensor interface and a sprayer interface. Such a unit may be added to existing toilets (or optionally built into new toilets) in a variety of ways. some examples are shown in FIGS. 8A through 8D. FIG. 8A shows a wall mount version, FIG. 8B shows a top of toilet mounting position, and FIG. 8C shows a system 150 configured to mount on the lower toilet structure behind the seat. FIG. 8D shows a free standing tank arrangement of system 150, which may be hung on a bracket as shown in the figure or placed on the floor as will be shown below.

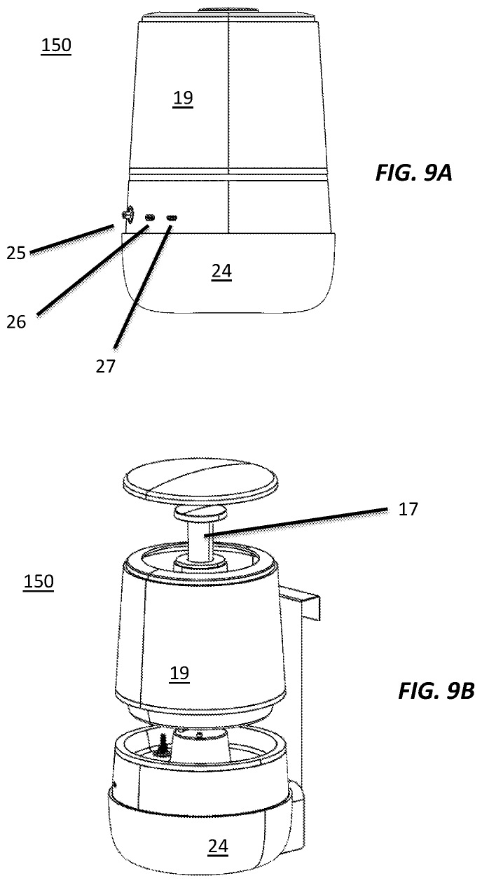

Beginning in FIG. 9A a variety of implementation details of cleaning system will be shown. By way of example the stand-alone tank embodiment, resting on the floor is shown, but the discussion applies to all packaging versions.

In FIG. 9A, the system includes a reservoir 19 attached to a base 24, which may include power supply 9. As shown in FIG. 9B, reservoir is removable from base 24. Additive pod 17 is installable and removable in reservoir/base structure.

External interfaces required include sprayer feedline port 25. Other electrical ports 26 and 27 are required. In particular an electrical port for connection to a flow sensor is needed. Depending on how the system is powered other electrical ports may be needed. Many bathrooms are not build with electrical power conveniently located near toilets. Thus one common embodiment is for power supply 9 to be a rechargeable battery, and the system 150, or at least the base may be moved to a location with power to connect a battery charger. For some bathrooms, with toilet adjacent power, it may be desirable to plug in the system 150 locally, so power supply may be direct AC or DC by way of an external converter to external AC. So electrical ports for power may vary accordingly. Also it may be desirable to treat local interfaces, such as the sprayer or other functions such as lighting, as data links, so one or more electrical ports 26 or 27 may be a data port such as USB.

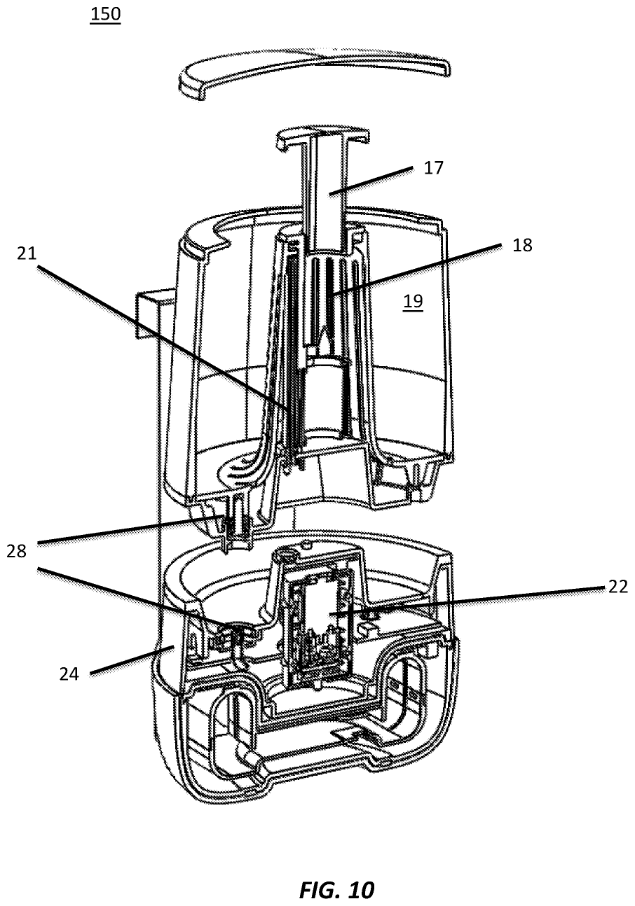

FIG. 10 shows more implementation details. In the embodiment shown Reservoir 19 includes fluid accessible mounting for electrodes 21 and pod port 18 for removable pod 17, which will be discussed in more detail below. Base 24 includes pump 22, and base/reservoir connections 28 which include both sealed electrical connections to indicate reservoir is installed and fluid interconnections to pump.

FIGS. 11A and 11B show details of an additive pod embodiment. In the embodiment shown, pos 17 contains with an additive solution, which is sealed into the pod b 17 by a foil seal 30 which may be pierced to open. When pod 17 is pushed into pod port 18, the foil is pierced by pierce point 29, allowing the pod fluid to drain into the reservoir 19. Electric connection 31 is activated when pod 17 is inserted, alerting controller that a pod is present or absent. Connection 31 may be placed above fluid fill line in some embodiments. Other components 20 may be interfaced to the controller such as fill sensors, control valves and the like may be present in some embodiments.

The produced electrolyzed water has a limited lifetime, as they break down over time. Also every electrolysis operation uses up pod additive. Thus pod replacement notification may be a function beneficially handled at the server level or at the PED level. The electrical connection 31 tells the controller when a pod has been installed and/or removed, and the controller will know when electrolysis has been performed. This information can be communicated to the controller and used to ensure proper pod use in a variety of ways.

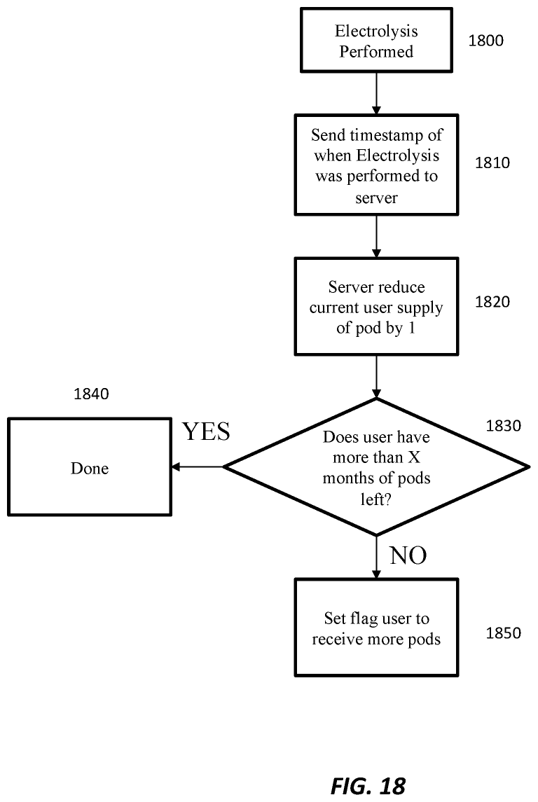

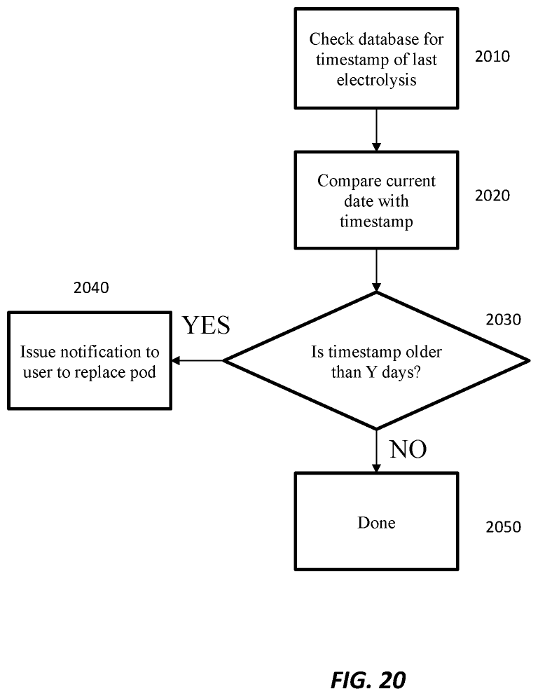

The controller may make use of time stamping pod installation and determining time of pod installed to determine if pod the pod needs to be replaced, and or monitoring electrolysis cycles to determine if a pod needs to be replaced. In either case, pod replacement alerts may be sent at either the user level or server level or both. In the case of server level notification, the server based application may initiate pod delivery to the user automatically. Both of these scenarios, which may operate in parallel are shown in flow chart form in FIGS. 18 and 20.

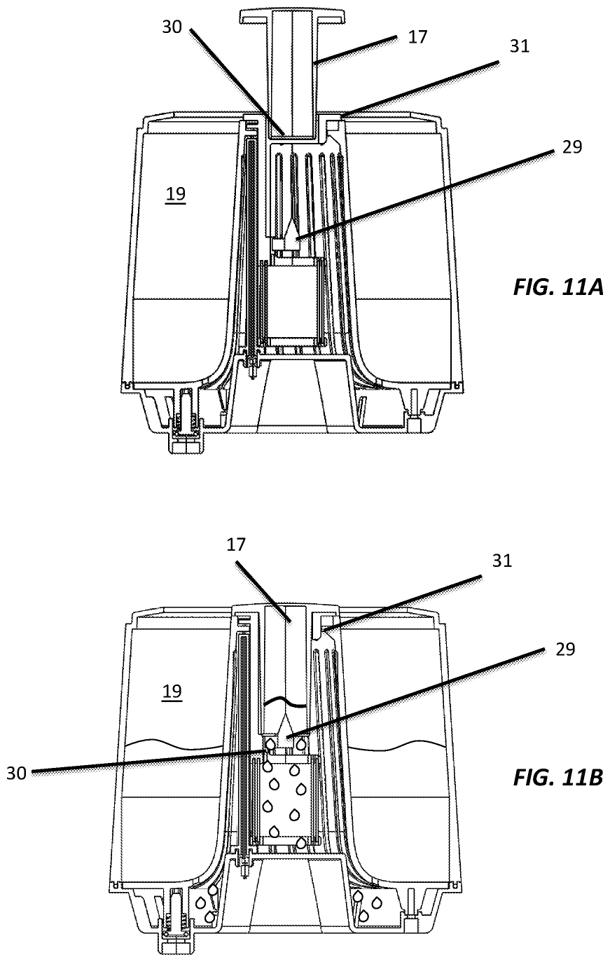

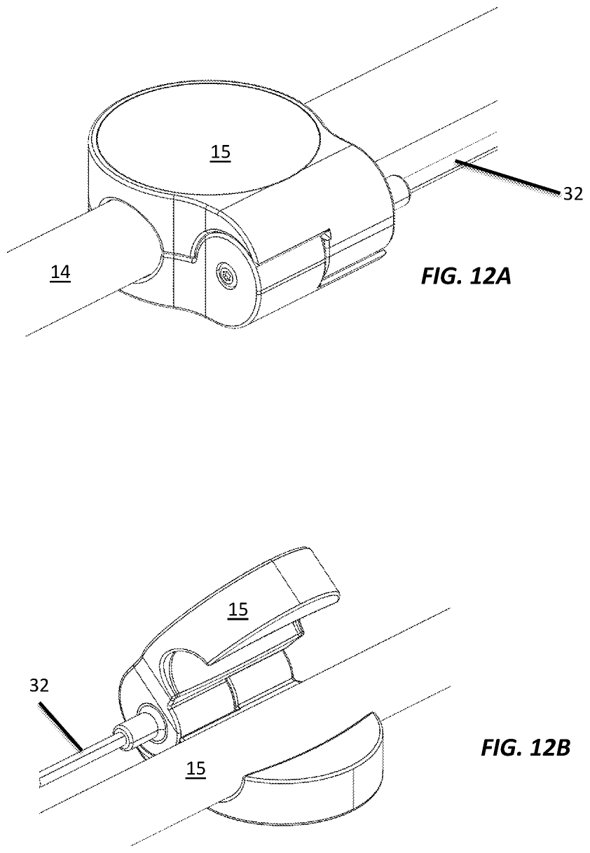

An embodiment of a flow sensor 15 is shown in FIGS. 12A and 12B. In this embodiment sensor 15 is an accelerometer held in proximity to a water line, such as toilet feedline. The sensor is clamped around the water line in the embodiment shown, but other arrangements are possible. The flow sensor 15 is connected to the toilet cleaning system by electric cal cable 32. The sensor may be used to alert the controller to the toilet flushing in order to initiate spray cleaning. However alternate flow detection capabilities and subsequent actions, with a flow sensor whose data is accessible over a network are possible, in particular, actions associated with toilet use and leak detection.

An optional additional sensor is a seat/lid status sensor 16, which again in some embodiments may be an accelerometer type sensor or other type of position switch sensor. Sensors may communicate data either wired or wirelessly or any combination thereof. In particular a wireless sensor package could operate off a coin cell battery and last approximately 1 year of normal use. The electronics could designed to be in ultra-low power deep sleep when no sensor activity is occurring. When the accelerometer is excited, it could initiate wake up from sleep mode into normal operating mode (which consumes more power) to take and communicate the data.

As described above, the sensors interface to the controller which can provide flow/position information and user interface functions across the network interface to either user devices in proximity, such as user PED's over a network such as Bluetooth as well as provide data to and receive data from servers 7 on the Internet (cloud 8), as well as other devices over the internet.

It is contemplated that in some embodiments, sensors 15 may be clamped to supply line 9 in a convenient tool-less fashion as shown. Other attachment means are possible

A calibration mode for the supply line sensor could operate as follows. During set up, the accelerometer could be calibrated via a user device application to “learn the toilet” as exemplified by the following steps. The accelerometer can measure the time water is flowing from start to stop. Several flushes could be measured to take into account variations in flow based on other plumbing activity. Water line pressure may range from home to home also. During calibration mode, the accelerometer may measure timing of flow on and flow off. During this flush calibration period, the accelerometer is sending data from the sensor to the cloud for analysis and then sending data back to the user via the app. In calibration mode, the length of flush, and/or vibrations of flush are recorded. After basic calibration is complete, the system establishes a “standard” fill time.

A user not fully flushing and/or less than standard fill time is measured may not be critical, but is still a variation in flow that does need to be accounted for. The main reason for reporting longer than standard fill time is to alert the user of plumbing leaks that could cause flood, damage, or just to prevent wasting water.

If the accelerometer picks up long flush signal or other signature of a leak, user settings can be set to control alarms and notifications to user.

The calibrated flow monitoring along with the connectivity enables many beneficial operating modes/capabilities:
Family traveling and the house will be empty for 1 month. User can go to settings mode and change to “Vacation mode” to report any and all flush or activity as heightened alert mode.
Customized alarms—user can add alarm setting to send alarm to specified users—example—if leaking valve past specified timing or wave form, text, email, or other method of communication, will be sent to head of household, or a list of phone numbers designated by user setting.
“Normal Use Mode” can be set to report of any accelerometer readings outside of flush calibration. For example, an internal valve leak, causing continuous flow of water, could be flagged as a high priority alarm and the system could text the user or follow other protocol based on user settings.
“Remote Caretaking” Retirement homes would like to track and provide user data to confirm a patient is using the toilet at standard intervals. Or users could install the monitoring system on an elderly family member's toilet and get daily texts to confirm if she is recovering from a surgery etc.
“Water conservation mode” during calibration mode, there can be an extra step to measure the actual water in gallons per flush. This may require additional inputs, such identification the actual toilet model and tie into a database to allow user to select from a list of toilets and each toilet on the list would have known number of gallons per flush. Water conservation mode could report daily, weekly, or monthly on actual number of flushes and water consumed, including leak detection and water usage.
“VRBO” or rental mode can be set to provide basic statistics to report to owner—Example—user owns 5 vacation rental units—all units have 3-5 bathroom per unit—The system application can have setting for owners to provide visibility to bathroom use such as if a unit is having a party—the owner will see higher than usual flush numbers and will have visibility on use. in general, the system can help owners understand how a facility is getting used, for example 1 bathroom has only 1 flush over 1 week period, vs another bathroom with 20 flushes, the owner can use this info to direct cleaning services to clean the higher used bathroom more than the other. System data can also be used to trigger cleaning services or provide a convenient way to determine if guests are in the facility vs vacant.
“Party detection mode” Parents away, and higher than normal flush rates are being seen. Settings can allow user to be notified if flush count exceeds normal allotment. The system could also be configured to provide use and/or excessive use information across the net through third party web based services including social media applications.

It is envisioned that in most embodiments, the system communicates to the cloud via a WiFi connection or by other means. The raw data may be sent to the cloud for computation on the back end. This allows a server based subscription service to manage the data using algorithms and calculations to optimize the system and learn and update baseline operating conditions over time. An example would be, during times of drought with the need to conserve water, city or state utility services could incentivize customers by providing a discount or rebate by having data to show that they do not flush during urination to save water (as an example). A server and/or local based system monitoring flow would provide the data over time, such a user could show data reports of quantity per flush per day, week or month before and after conservation methods were used.

In general the system supports both the implementation of a cloud based subscription as well as local user data acquisition and monitoring through local network connectivity. Push alerts either local or via the cloud could be employed for critical information such as leak detection.

The system can, for example via an open API, integrate with other devices such as activity trackers, hydration monitoring, wellness monitoring, diet monitoring, health condition recovery etc. And of course tie in to other smart toilet components such as weight monitoring as described above.

An additional lid/seat sensor, possibly also an accelerometer, or other position switch type sensor, could be employed in tandem or independently of the flow sensor.

In some embodiments, a toilet system service provider may reside at the server level in the cloud or internet. This system level service could include a variety of health monitoring tracking and information based on correlating ID information with toilet use over time. Overall usage could also be monitored and bathroom supply material consumption could be estimated, and either alerted to users or automatically ordered. In affect a cloud level subscription service, which may include self ordering, auto-fulfillment or other automated features centered around data provided by the system is made possible, for items related to bathroom use, or other suitably related items such as toothpaste, toilet paper, shampoo, food, drink and the like. These functions could also be handled at the user device level with appropriate applications running on user PED's

Other intelligent toilet features could operate in tandem with the flow sensing and/or seat/lid position sensor infrastructure. In particular in a toilet with smart connected features such as seat heating and cleaning/deodorizing functionality, the supply line flow sensor and/or the seat position sensor could provide the information to initiate or end other smart toilet functions.

In one embodiment, the flow sensing function could in tandem with a weight sensing toilet seat as described above and or seat position function to determine if the toilet was flushed after use and provide appropriate user indications to the system controller to pass on to users. In tandem with an auto-flush mechanism under control of the smart toilet system, a hands free flush after use capability could be implemented.

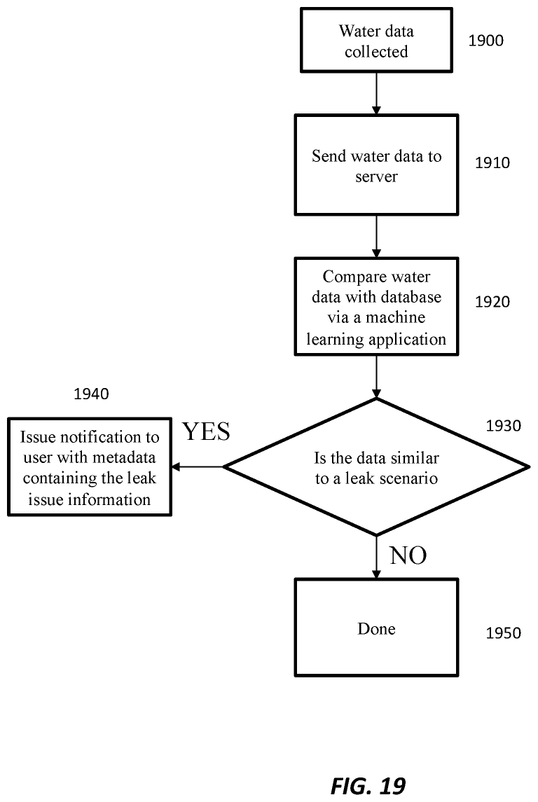

Leak detection is a particularly useful application of a flow sensor interfaced to a controller and through the controller to a server based application. if a leak condition is detected, notifications and actions at the server level may include leak detection alerts, leak detection recommendations depending on type of leak determined by calibrating the flow sensor, and even server application initiation of automatically sending proactively a repair kit to replace toilet components. Such leak detection operations are shown in flow chart form in FIG. 19.

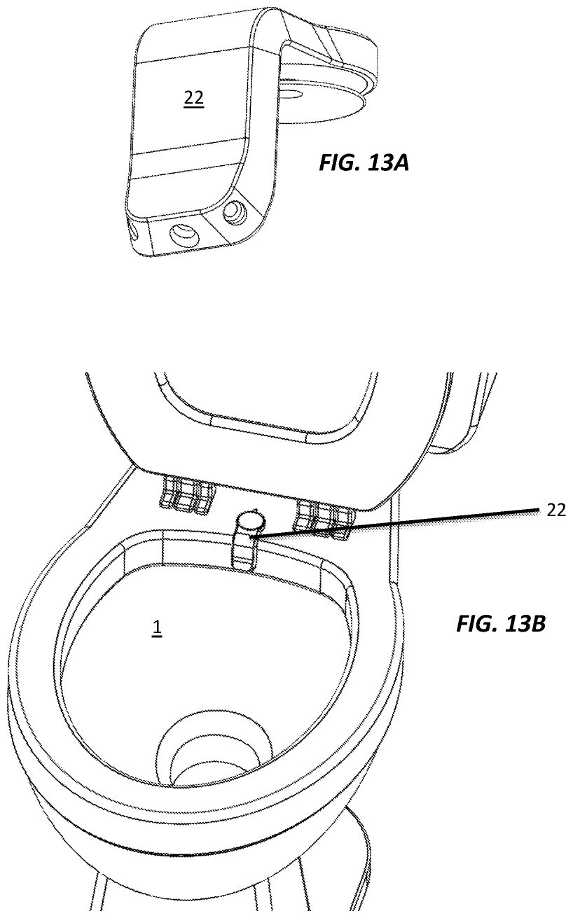

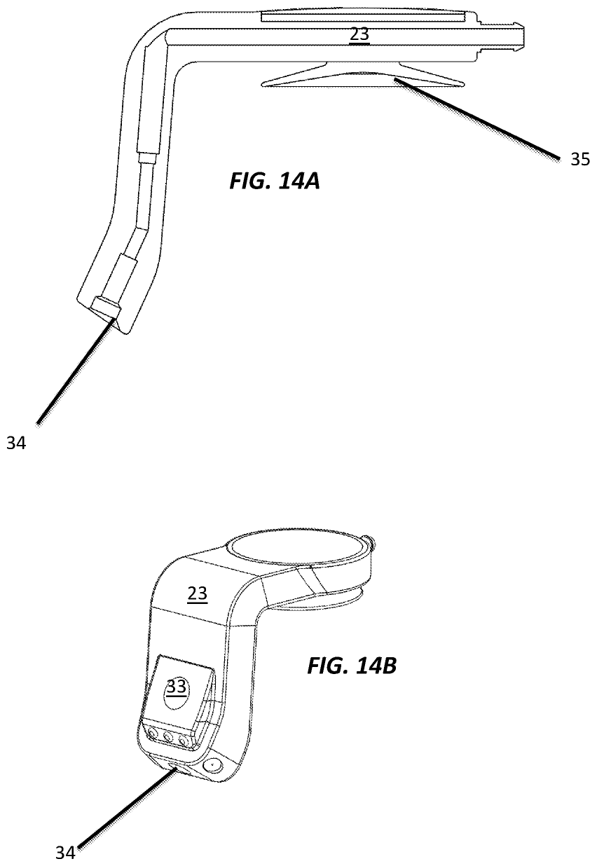

FIGS. 13A and 13B show details of a sprayer 22, which is attachable to toilet 1 and configured to spray electrolyzed fluid from the electrolyzer for the purpose of cleaning the toilet bowl. FIGS. 14A and 14B show more sprayer 23 implementation details, including sprayer nozzle 34, and other optional sprayer functionality embodiments such as lighting elements 33. Also shown is a sprayer mounting element 35, which is a suction cup element in the Figure but obviously other mounting provisions are possible.

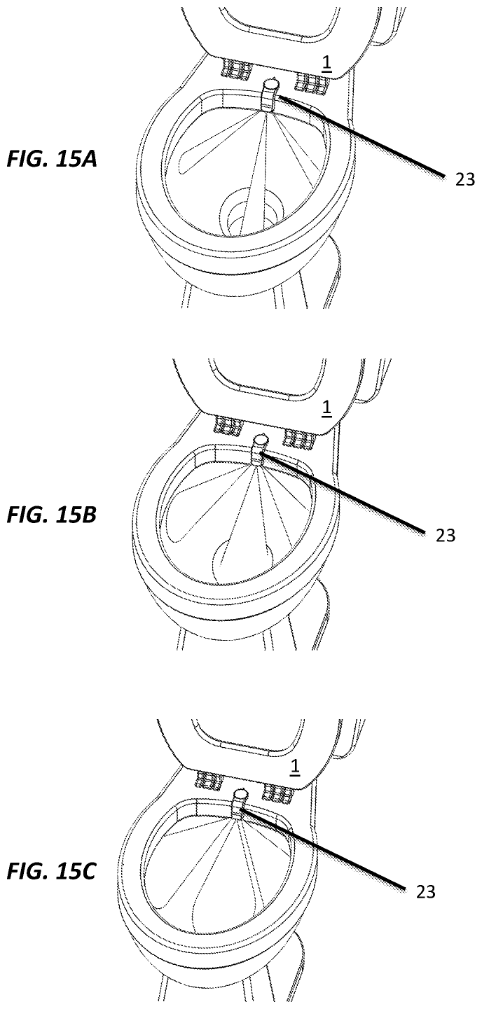

FIGS. 15A, 15B, and 15C, show sprayer embodiments with three nozzles disposed to aim around the toilet bowl with sprayer attached and set at various angles with various spray cross-sections to cover the entire bowl when activated.

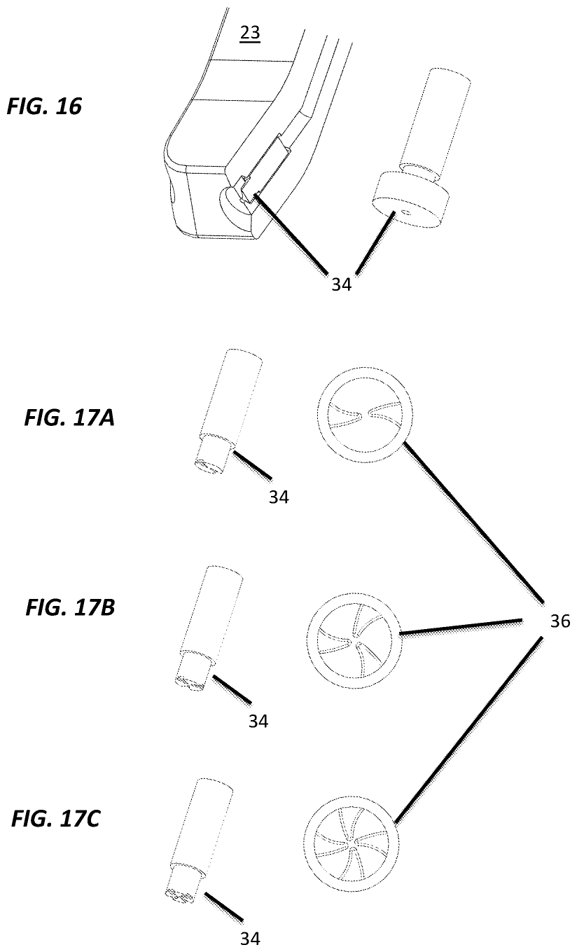

FIG. 16 shows nozzle 34 mounted into sprayer body 23. FIGS. 17A, 17B, and 17C show various nozzle 36 orifice texture patterns, which have been shown to achieve beneficial spray patterns. In particular, partially depending on the orifice pattern chosen, the spray may be configured to be a liquid spray or a mist depending on the pressure applied by the pump, with lower pressure achieving a liquid spray, and higher pressures achieving a mist spray. The pump may be configured to operate at different pressures (via pump speed control, pump power applied, or other means) to achieve mist or liquid spray, or possible both during one electrolyzed cleaning cycle, as desired.

Various use and operational aspects of the smart networked toilet, including cleaning system, in the context of operation with a server based toilet service application will be discussed below. The following discussion includes the assumption of one or more features implemented, including controller interface and control of sensors and actuators, including battery management information, networked/local user interface through apps running on PED's, and/or dedicated controls/displays, and a server based toilet/bathroom management service interfaced to the bathroom/toilet controller. Some of these options are described in detail above, while others are other possible implementations extending the concept of a complete networked bathroom ecology capable of operating in a variety of modes. Some details described below are directly associated with embodiments shown above, while others apply to embodiments that may be contemplated within the overall implementation of networked bathroom/toilet enhancements

Additive pods and fluid reservoirs are contemplated to contain substances, likely in fluid form, that are suitable to make the experience of using the toilet more pleasant and healthier. Such substances may include electrolysis additives for mixing with water solutions or other cleaning fluids, as well as scent based substances including deodorizers and aromatherapy oils. The reservoirs could optionally include level sensors reporting back to the controller 2 so that consumption is monitored and refilling indications can be made. Reservoirs and/or feedlines may also be combined with atomizing elements including, electrolyzers, heaters, electrostatic or suitable nozzles or pores to convert the fluids such as deodorizers to a mist or vapor.

A variety of configurations are possible. A single pump and single feedline coupled with processor controlled valves may be used to select a desired combination of fluid delivery. Or dedicated feedline/pump/reservoir arrangements may be made. For instance the cleaner fluids may be advantageously delivered to the toilet in a way that swirls the cleaner around the bowl. This would work as well for scents, but it may be desirable to deliver atomized scents differently than the cleaning solution for some applications

It is contemplated that in some embodiments, the processor may be configured to control the pump(s), actuate the reservoir valves and/or electrolyzer, monitor the reservoir fill sensors and communicate to the outside world by way of wired, wireless, and/or networked means. The cleaning module can be configured as an after market upgrade to an existing toilet/toilet seat combination, configured to work in concert with a compatible toilet seat design or built in as part of suitably designed new toilet

The cleaning module may be battery or wall powered. Wall power may be problematic as most bathrooms do not place power outlets adjacent toilets for safety reasons. However the increased adoption of powered toilets (bidet units for example) is leading to more houses being designed with suitably protected outlets in the toilet area, or such outlets may be added to existing houses. However it is contemplated that most cleaning systems and other devices will run off of battery power. The batteries in some embodiments will be removable and/or rechargeable. In some embodiments the controller will access, monitor and/or report battery charge

The cleaning/odorizing functions may be controlled in a variety of ways. For the case where the cleaning module is part of smart toilet system including other sensors, the information that a toilet is about to or has been used could come from these other sensors, including for example, weight sensors, seat position sensors, sound/motion sensors and others. The user may inform the module of their presence by way of a direct user interface, voice activation, or wirelessly via personal electronics devices running an application that accesses the system controller. For the embodiments where the module is part of a server based smart toilet system, the control functions and data reporting may be accessible remotely across the internet or other network.

Cleaning/deodorizing may be initiated in a variety of fashions. Automatically after and/or before toilet use, detected either by way of a flush detection capability and/or weight on the seat detection, Bluetooth based presence detection, IR presence detection, sound detection, vibration detection in floor, or directly initiated by the user. Scheduled operations or remotely initiated operations are possible. Basically whatever combination of the specific fluids delivered, the time they're delivered, the quantity delivered, and so on can be programmed, configured for personal preference by identifying users electronically or through the use of other, as well as by voice recognition.

For the cleaning system embodiment shown above embodiment, the reservoir when lifted may actuate a check valve to seal the tank port. Also lifting the tanks to remove or refill also disconnects an electrical connector such as pogo pins, which both may be used to disconnect control and power signals to pump in the unit base as well as indicate to the system controller the status of the tank.

One or more removable active ingredient pod residing in a central shaft may include electrolyzing deodorizing, cleaning and/or other cleansing and aromatic compounds releasable through openings in the shaft walls into the fluid reservoir.

Pogo pins may be located above the fill level of the tank to improve electrical reliability. In some embodiments it may be desirable to include electrodes in or in line with the feed line to the electrode to electrolyze all or part of the fluid to accentuate the cleaning and/or deodorizing effectivity. Electrolysis could also be accomplished with a separate reservoir specifically for electrolyzed fluid, which could also have separate active ingredient pods as shown in above described embodiments associated with electrolysis action.

It is envisioned that in some embodiments, the system communicates to the cloud via a WiFi connection or by other means. Usage and material consumption data may be sent to the cloud for computation on the back end. This allows a server based subscription service to manage the data using algorithms and calculations to optimize the system and learn and update baseline operating conditions over time. For example, in the absence of other toilet sensors, the usage data from the cleaning module could be correlated with data such as water usage and individual user behavior patterns.

In general the system supports both the idea of a cloud based subscription as well as local user data acquisition and monitoring through local network connectivity. Push alerts either local or via the cloud could be employed for critical information such lack of use for an elderly or incapacitated individual.

The system can, for example via an open API, integrate with other devices such as activity trackers, hydration monitoring, wellness monitoring, diet monitoring, health condition recovery etc. And of course tie in to other smart toilet components such as weight monitoring and flush detection.

In some embodiments, a toilet system service provider may reside at the server level in the cloud or internet. This system level service could include a variety of health monitoring tracking and information based on correlating ID information with toilet use over time. Overall usage could also be monitored and bathroom supply material consumption could be estimated, and either alerted to users or automatically ordered. In affect a cloud level subscription service, which may include self ordering, auto-fulfillment or other automated features centered around data provided by the system is made possible, for items related to bathroom use, or other suitably related items such as toothpaste, toilet paper, shampoo, food, drink, and the like. These functions could also be handled at the user device level with appropriate applications running on user PED's

Other intelligent toilet features could operate in tandem with the cleaning module infrastructure. In particular in a toilet with smart connected features such as seat heating and flow sensing functionality, could provide the information to initiate or end cleaning module functions.

In some embodiments the cleaning device may be triggered by the detecting the proximity of a network enabled device such a smartwatch or smartphone entering the vicinity of the toilet. For example for bluetooth enabled devices an RSSI reading may alert the toilet to the presence of a user, and various options such a clean on approach and/or clean on departure may be enabled. As mentioned above, for toilets including both a cleaning device and seat position sensor the sensor position and/or position history could be used to trigger cleaning operation. Another approach would be to use an optical level sensor installed in the toilet bowl, toilet tank, and/or in the seat or any suitable location to monitor water level and determine flush or fill from water level.

A variety of use case scenarios may be contemplated for a cleaning/deodorizing accessory with or without other smart toilet accessories. These scenarios are optional, and not all systems will enable all options. The following is intended to provide a range of possible operational aspects. not all of which are possible with any given system implementation.

Optional Set-up scenarios

As a user with no experience replacing a toilet seat, a user should be able to have access to step by step guidance

As a user with no experience replacing, installing, or setting up a IOT device, a user should have access to a real person to walk them through the experience.

A user should have no problem connecting the device to the wifi or other networks using similar patterns that are familiar for connecting personal devices such as phones to networks,

A user should have clear instructions on how to remove a current toilet seat and how to screw in the new toilet seat.

A user should have clear instructions on how to hook up the cleaning sprayer and power connections and flow detection kit

A user should be guided on activating the first supply of cleaning solution

A user should be asked if a screw driver is available for installation on checkout and if not, a screw driver should be sent free with the packaging.

The seat should be easy to install and should look good on a user's toilet whether it is elongated or round.

Optional Cleaning Scenarios

An end user should be notified about need to replace additive solution pods.

An end user should be notified when to order a new cleaning pod.

An end user should be able to subscribe to have cleaning pods automatically sent when needed.

An end user should be able to remotely clean the toilet through a PED application.

An end user should be able to remotely clean the toilet through Alexa, SIRI or the like.

If an end user attempts to clean the toilet and there is no cleaning solution left, the user should receive a notification and an option to get more cleaning pods

An end user should be able to tune how long the cleaning spray actuates.

If an end user is not subscribed to auto fulfillment of additive pods, they should see the option to manually buy a single pod or subscribe to auto fulfillment service.

Optional Multiple Device Management

An end user should have a UI that enables them to manage multiple toilets at once.

An end user should be able to order additive pods for all their toilets at a single time

An end user should easily be able to see all the cleaning levels and statuses of toilets that they own in a single glance

An end user should be able to see the savings they could have realized if they choose to use auto fulfillment vs one off fulfillment

Optional Power & Battery Scenarios

An end user should see the battery level and an estimated number of days left till need to recharge

An end user should be alerted when there is 1 day left of battery

An end user should be alerted when there is no battery

If a battery remains uncharged for 7 days, an end user should be contacted by customer support to find out why provide assistance.

An end user should be able to activate and tune energy savings from a PED application and get feedback on how the tuning effects the overall battery life

An end user should be able to upgrade to have a longer battery life.

If subscribed to auto fulfillment, an end user should receive a free replacement battery when the battery becomes old to remain an active user.

Optional Water Monitoring Scenarios

An end user should be able to see both individual toilet water usage as well as a summary.

An end user should see historical water usage estimates in both gallons and dollars.

An end user should see content with tips on how to reduce water bill.

An end user should receive a notification if a leak is detected with an estimate of how much water per day the leak is wasting in both gallons and dollars.

An end user should receive a notification if an overflow event is detected.

If a leak is detected an end user should be recommended local plumbers that can service the leak.

If a leak is detected there should be a status that shows on both the app and toilet LEDs.

If a leak was detected and now the status is resolved, the alert should dismiss and leak status should dismiss.

An end user should be able to dismiss the leak alert manually from the app in case there is a false alert. The algorithm should detect this for better filtering of alerts.

The leak detection should calibrate with a user's specific toilet to get as accurate readings as possible.

In the event of a detected leak, the user should have the option of automatically receiving a repair kit shipped by the server level application.

Optional Heated Seat Scenarios

An end user should be able to adjust the desired heated seat level.

An end user when adjusting heat settings should understand the consequences of battery life to changing those heat settings and should get a new projection on battery life.

The default heat settings should be tuned so that the battery lasts at least one month.

When a user lifts up the lid but leave the seat down, the heat should heat up by default.

When a user lifts both the lid and seat, the heat should not turn on so that the battery last longer.

An end user should be able to heat the seat remotely.

An end user should be able to command seat heating from a PED application, either on command or upon detection of an approaching app enabled PED.

The seat should know when an end user sits down to use the toilet and when they stand to use the toilet, so that if the seat is to heat when the user approaches, it can heat intelligently to can save power.

The seat should heat quickly.

The heat should turn off by default when a user stands up.

Optional Toilet Seat open and close Scenarios

An end user should know when the lid and seat are up or down so that the user can know who keeps leaving the seat up.

An end user should be able to remotely close the lid.

An end user should be able to remotely open the lid.

An end user should be able to have the lid close automatically when they approach.

The lid should automatically close after a user stands up and flushes the toilet.

The seat should have a slow close design.

Optional Wellness Scenarios

An end user should be guided through the weight tracking calibration.

An end user should only have to do the weight tracking calibration once.

An end user should see a line showing my weight trend.

The weight trend line should be thick enough so that the weight margin of error doesn't confuse a user.

An end user should have the paid option to see unlimited weight history

An end user should have the paid option to export data to any exercise partner.

If a user pays for automatic fulfillment service, they should get to have the full weight premium features for free.

An end user should be able to see the frequency of use.

An end user should be able to monitor the average length of use.

An end user should have a paid option to get insights from their usage patterns.

Optional User Management Scenarios

A household should be the main account.

Sub users can be under one account and can be given access to control toilet settings.

Each sub user should have their own weight data.

The household account should be tied to one subuser account at the admin.

The admin can be reassigned to any subuser account.

Optional Privacy Scenarios

Users can choose to turn off weight reporting to the cloud and weight data will not be recorded.

Optional Removal Scenarios

An end user should be able to deactivate a toilet in case the toilet is sold to someone else.

If a user attempts to activate a toilet assigned to an existing account, an email can be sent to the account holder to reassign.

If a user attempts to activate a toilet assigned to someone else, they can contact customer support and they should be able to remove that toilet from the account.

Depending on the embodiment, certain acts, events, or functions of any of the processes described herein can be performed in a different sequence, can be added, merged, or left out altogether (e.g., not all described acts or events are necessary for the practice of the algorithm). Moreover, in certain embodiments, acts or events can be performed concurrently, e.g., through multi-threaded processing, interrupt processing, or multiple processors or processor cores or on other parallel architectures, rather than sequentially.

The various illustrative logical blocks, modules, and process steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, the LUT described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, and Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or processes illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

### REFERENCE NUMBER DEFINITIONS

100 Electrolyzer
150 Toilet Cleaning System
1 Toilet
2 Processor/controller with network Interface
3 Sensor
4 Actuator
5 Additives
6 Personal Electronic Devices
7 Servers
8 Cloud
9 Power Supply
10 Toilet Seat
11 Weight Sensors
12 Gasket
13 Heater Sensor
14 Toilet Supply Line
15 Flow Sensor
16 Position or other Sensor
17 Electrolyzer Additive Pod
18 Pod Port
19 Reservoir
20 Fill Sensor/Valve
21 Electrodes (Anode and cathode)
22 Pump
23 Sprayer
24 Base
25 Sprayer feedline port
26 Electrical Ports
27 Electrical Ports
28 Base/Reservoir electrical connection
29 Foil Pierce-point
30 Pod Foil
31 Pod Installed Electrical Connection
32 Flow Sensor Electrical cable
33 Lighting
34 Nozzles
35 Sprayer Attachment
36 Nozzle Orifice Patterns

We claim:

1. A method for operating an intelligent toilet system, the system including:
a liquid reservoir with control valve in a housing configured to be placed externally to a toilet;
a controller, configured to be placed externally to the toilet including as part of the reservoir; housing;
a power supply, configured to be placed externally to the toilet including as part of the reservoir; housing
electrolyzer electrodes in fluid communication with the tank and interfaced to the controller;
a pod mount for replaceable cleaning material pods in fluid communication with the tank and electrically interfaced to the controller;
a fluid level sensor in the tank electrically interfaced to the controller;
a clamp-on flow sensor configured to clamp on the toilet feedline and electrically interfaced to the controller;
a pump in fluid communication with the tank and electrically interfaced to the controller;
a sprayer connected fluidly to the pump, configured to mount externally in a position to spray the toilet bowl;
at least one of a wireless or wired interface between the controller and a network.
an app configured to execute on a user device, wherein the user device is connectable through the network to the controller;
the controller executing steps comprising;
monitoring the water level of the tank, and communicating at least one of water level or need to refill tank to the user device app over the network;
monitoring the pod mount to determine the presence of a pod and the time that a pod is mounted, and notifying the user device app over the network if the pod needs to be replaced due to length of time installed;
monitoring the flow sensor signal;
using the flow sensor signal to determine if the toilet is flushed, and if pod presence and water level are suitable, activating the electrolyzer and pump;
using the flow sensor signal to monitor at least one of fill time or steady state flow and using at least one of these parameters determine changes over time that indicate a leak, and communicating leak status to the user device app over the network;
communicating need to change pod to the user device app over the network based on number of electrolyzer cycles;
activating the electrolyzer and pump in response to communication from the user device app over the network.

2. The method of claim 1, further including reporting system activity including electrode and pump activity to a server

3. The method of claim 2, wherein the server automatically reorders more cleaning pods based on usage data received

4. The method of claim 1 wherein the reservoir housing is configured to at least one of;
sit on floor adjacent to the toilet,
hang from toilet body by means of a hanging bracket,
mount to wall adjacent toilet,
sit on top of toilet,
or placed on counter top or other fixed horizontal surface

5. The method of claim 1, wherein the cleaning materials pod comprises a foil sealed vessel, which when installed in the reservoir, the foil is pierced by a pin allowing the cleaning materials to drain into the reservoir.

6. The method of claim 1, wherein the sprayer at least one of clips, sticks via adhesives, is held by suction to the toilet, or is mechanically connected to the toilet.

7. The method of claim 1, wherein the sprayer comprises three nozzles at differing angles configured to deliver a fine spay mist wherein the mist covers the entire inside of the toilet bowl, wherein to deliver the fine mist, the fluid moves through the sprayer, into the nozzle, through textured orifices, and finally exiting through the nozzles.

8. The method of claim 1, wherein the sprayer is user adjustable to adjust the spray via the user app to increase or decrease power to the pump wherein lower power results a stream of fluid for a point contact of the fluid to the bowl and higher power creates more pressure resulting in a fine mist.

9. The method of claim 1, wherein the power supply comprises at least one of:
a battery; or,
a plug-in power source.

10. The method of claim 1, wherein the flow sensor comprises an accelerometer.
